(19) United States
(12) Patent Application Publication
(10) Pub. No.: US 2005/0031713 A1
(43) Pub. Date: Feb. 10, 2005

(54) Methods for administering active agents to CYP3A4 sensitive patients

(76) Inventors: Elliot Ehrich, Lincoln, MA (US); Trevor Mundel, Carlisle, MA (US)

Correspondence Address: Carolyn S. Elmore;Elmore Craig, P.C., 209 Main Street, Chelmsford, MA, 01863, US

(21) Appl. No.: 10/635,234

(22) Filed: Aug. 6, 2003

(51) Int. Cl. A61K 35/78; A61K 31/554; A61K 31/704; A61K 31/7048

(52) U.S. Cl. 424/736; 514/211.07; 514/282; 514/37; 514/29; 514/253.07; 514/317

(57) ABSTRACT

The present invention relates, in part, to the discovery that parenterally administered extended release formulations possess an unexpected advantage in treating patients possessing active CYP 3A4. This advantage is particularly beneficial where the individual is concomitantly administering a CYP 3A4 inhibitor or is in risk of doing so. Thus, the invention relates to a method for treating individuals possessing a functional CYP3A4 gene with an active agent metabolized by CYP3A4 comprising parenterally administering the active agent in a first extended release formulation in a first administration and the formulations for use in such methods. The invention further includes a method for preventing adverse drug reactions in individuals possessing a functional CYP3A4 gene with an active agent metabolized by CYP3A4 comprising parenterally administering the active agent in a first extended release formulation in a first administration.

### BACKGROUND OF THE INVENTION

Polymorphic cytochrome P450 (CYP) has been identified as being important in the metabolism of many drugs. Indeed, CYP 3A4 has been linked to the metabolism of a number such drugs, such as clozapine and aripiprazole. Individuals have been genotypically classified for CYPs. Scordo et al., Psychopharmacology (1999) 147:300-305 and Leyland-Jones U.S. Patent Application Publication 2003/0072710, which are incorporated herein by reference in its entirety. There exists a need for improved methods of delivering active agents, such as drugs and medicaments, which maximize the pharmacological profile of the active agent.

### SUMMARY OF THE INVENTION

The present invention relates, in part, to the discovery that parenterally administered extended release formulations create an unexpected advantage in treating patients possessing an active CYP 3A4 enzyme. This advantage is particularly beneficial where the individual is administering a CYP 3A4 inhibitor or is at risk of administering a CYP 3A4 inhibitor.

Thus, the invention relates to a method for treating individuals possessing a functional CYP3A4 gene with an active agent metabolized by CYP3A4 comprising administering the active agent in a manner which bypasses first-pass metabolism. In a preferred embodiment, the invention relates to the administration of a first extended release formulation in a first administration and the formulations for use in such methods.

The invention further includes a method for preventing adverse drug reactions in individuals possessing a functional CYP3A4 gene with an active agent metabolized by CYP3A4 comprising parenterally, e.g., by injection, administering the active agent in a first extended release formulation in a first administration.

### DETAILED DESCRIPTION OF THE INVENTION

Thus, the invention relates to pharmaceutical compositions or extended release formulations for use in the treatment of individuals possessing a functional CYP3A4 gene with an active agent metabolized by CYP3A4 and the methods of treating such individuals comprising administering the active agent in a manner which bypasses first-pass metabolism. In a preferred embodiment, the invention relates to the administration of a first extended release formulation in a first administration and the formulations for use in such methods. The invention further relates to a method for preventing adverse drug reactions in individuals possessing a functional CYP3A4 gene with an active agent metabolized by CYP3A4 comprising parenterally administering the active agent in a first extended release formulation in a first administration.

The active agent for use in the inventions can be any active agent that is metabolized by CYP 3A4 and includes drugs, medicaments, diagnostic agents, neutraceuticals, etc. Preferred active agents include those that are active in the central nervous system (or CNS), such as those requiring transport across the blood brain barrier and for treating individuals under psychiatric treatment. Examples of such drugs include antipsychotics, antidepressants, serotonin reuptake inhibitors, neuroleptics, and opioids.

Examples of substrates for CYP 3A4 include aripiprazole, clozapine, rifampin, progesterone, propranolol, trazodone, prednisone, quinine, nitrendipine, ritonavir, R-warfarin, quinidine, taxol, ondansetron, nisoldipine, nimodipine, nifedipine, nicardipine, salmeterol, nelfinavir, triazolam, paclitaxel, verapamil, zolpidem, zaleplon, pravastatin, pimozide, haloperidol, caffeine, zileuton, terfenadine, vinblastine, saquinavir, methadone, testosterone, nefazodone, temazepam, tamoxifen, tacrolimus, simvastatin, sildenafil, sertraline, vincristine, chlorpheniramine, miconazole, dexamethasone, dapsone, cyclosporine, cyclophosphamide, cyclobenzaprine, codeine-N-demethylation, cocaine, clonazepam, clomipramine, clindamycin, diazepam, cisapride, diltiazem, cerivastatin, carbamazepine, cannabinoids, busulfan, buspirone, atorvastatin, astemizole, amlodipine, amitriptyline, alprazolalm, alfentanil, clarithromycin, frazodone, midazolam, mibefradil, lovastatin, losartan, lidocaine, lercadipine, lansoprazole, ketconazole, isradipine, indinavir, imipramine, dextromethorphan, hydrocortisone, navelbine, finasteride, fexofenadine, fentanyl, felodipine, etoposide, ethosuximide, estrogens, oral contraceptives (estradiol), erythromycin, dronabinol, doxorubicin, donepezil, disopyramide, ifosfamide and alfentanil and analogs thereof. A preferred active agent is aripiprazole. Similar drugs are described in U.S. Pat. Nos. 5,006,528 and 4,734,416, for example.

The active agent is preferably administered in an extended release formulation. In one embodiment, the extended release formulation releases the active agent over a period of at least about 7 days, preferably at least about 14 days, alternatively for at least 2, 3 or 4 weeks.

In one embodiment, the formulations can be administered as a single or sole dose. However, the invention is particularly beneficial for those individuals that require constant or chronic therapy, such as those that receive repeated doses over several weeks or months or more. In such dosing regimens, the method can comprise a first administration of a first extended release formulation and a second administration of a second extended release formulation. The second formulation can be the same, substantially the same or different as the first and can include the same active agent or a different active agent. For example, the second formulation can be administered at about 7 days, or more, such as at least about 14 days, or at least about 17 days, after the first administration, where the first administration results in the release of agent for a period of 1, 2, 3, 4, 5, 6, 7, 8, 9, 10, 11, 12, 13, 14 days, or more.

The term “therapeutically effective amount” is further meant to define an amount resulting in the improvement of any parameters or clinical symptoms. The actual dose may vary with each patient and does not necessarily indicate a total elimination of all disease symptoms.

As used herein, the term “individual”, “subject” or “patient” refers to a warm blooded animal, including but not limited to humans, such as a mammal which is afflicted with a particular disease state.

A therapeutically effective amount of the compound used in the treatment described herein can be readily determined by the attending diagnostician, as one skilled in the art, by the use of conventional techniques and by observing results obtained under analogous circumstances. In determining the therapeutically effective dose, a number of factors are considered by the attending diagnostician, including, but not limited to: the species of mammal; its size, age, and general health; the specific disease involved; the degree of or involvement or the severity of the disease; the response of the individual patient; the particular compound administered; the mode of administration; the bioavailability characteristic of the preparation administered; the dose regimen selected; the use of concomitant medication; and other relevant circumstances.

Preferred amounts according to the selected mode of administration are able to be determined by one skilled in the art. Pharmaceutical compositions can be manufactured utilizing techniques known in the art. Typically the therapeutically effective amount of the compound will be admixed with a pharmaceutically acceptable carrier.

The compositions of the present invention can be administered by routes that by-pass first-pass metabolism. First pass metabolism occurs most frequently upon oral administration. As such, suitable modes of administration can include inhalation or pulmonary administration, sublingual, buccal, transdermal, injection, implantation and other parenteral routes of administration. Preferred methods of administration include intramuscular and subcutaneous injection, for example.

For injection, the compounds may be dissolved in a physiologically acceptable pharmaceutical carrier and administered as either a solution or a suspension. Viscous injectable carriers are preferred, having for example, a viscosity of at least 20 cp at 20° C. In other embodiments, the fluid phase of the suspension has a viscosity at 20° C. of at least about 30 cp, 40 cp, 50 cp, and 60 cp. The composition may also comprise a viscosity enhancing agent, a density enhancing agent, a tonicity enhancing agent, and/or a wetting agent. Illustrative pharmaceutical carriers also include water, aqueous methylcellulose solution, saline, dextrose solutions, fructose solutions, ethanol, or oils of animal, vegetative, or synthetic origin. The pharmaceutical carrier may also contain preservatives, and buffers as are known in the art.

In another embodiment, the formulation can be surgically implanted. Such formulations can include any of the well-known biodegradable and bioerodible carriers, such as polylactides and poly-lactide-co-glycolides and collagen formulations. Such materials may be in the form of solid implants, sponges, and the like. In any event, for local use of the materials, the active ingredients usually are present in the carrier or excipient in a weight ratio of from about 1:1000 to 1:20,000, but are not limited to ratios within this range.

Preferably, the compounds are in an extended release formulation. Extended (also referred to as sustained or controlled) release preparations may be achieved through the use of polymers (preferably poly-lactide or poly-lactide-co-glycolide polymers) to entrap or encapsulate the active agent described herein. Extended release formulations can be made by spray drying polymer-drug mixtures, emulsion-based technologies, coacervation based technologies, film casting, extrusion based technologies and other processes to manufacture polymer-drug microparticles possessing an extended release profile. Examples of suitable extended release technologies that can be used to incorporate the agents herein include, without limitation, the MEDISORB® technology, as described in, for example, U.S. Pat. Nos. 6,264,987 to Wright, 5,654,008 and/or 5,792,477, for example; the PROLEASE® technology, as described, for example in U.S. Pat. No. 6,358,443 to Herbert; the technologies described by Southern Research Institute, as described for example in U.S. Pat. Nos. 5,407,609 and 6,306,425; and “Method of Preparing Sustained Release Microparticles,” U.S. application Ser. No. 60/441,946, filed Jan. 23, 2003, and the technologies described by Alza Corp., including the ALZAMER® Depot injection technology. The contents of these patents are incorporated herein by reference in their entirety.

In one preferred embodiment, the agent is present in the extended release device or formulation in an amount of at least about 5% by weight, preferably at least about 10% by weight, more preferably at least about 30% by weight of the total weight of the device, or formulation.

Also contemplated is the entrapment of the active agent in microparticles prepared, for example, by coacervation techniques or by interfacial polymerization (for example, hydroxymethylcellulose or gelatine-microcapsules and poly-(methylmethacrylate) microcapsules, respectively), in colloidal drug delivery systems (for example, liposomes, albumin, microparticles, microemulsions, nanoparticles, and nanocapsules), or in macroemulsions.

When the composition is to be used as an injectable material, including but not limited to needle-less injection, it can be formulated into a conventional injectable carrier. Suitable carriers include biocompatible and pharmaceutically acceptable solutions.

The individuals who can be beneficially treated by the described methods include those individuals who possess a functional CYP 3A4 gene and are at risk for drug interactions. Thus in one embodiment, the individual is one who is receiving or may be receiving a CYP3A4 inhibitor, such as fluconazole, omeprazole, saquinavir, quinine, ritonavir, nelfinavir, norfloxacine, sertraline, troleandomycin, diltiazem, delaviridine, nefazodone, zafirlukast, gestodene, amiodarone, cannabinoids, cimetidine, ciprofloxacin, clarithromycin, diethyldithiocarbamate, fluvoxamine, fluoxetine, mifepristone, grapefruit juice, indinavir, itraconazole, ketoconazole, metronidazole, mibefradil, miconazole, and erythromycin.

Alternatively, the method includes preventing CYP 3A4 drug-drug interactions in individuals at risk thereof comprising administering the formulations described herein.

A MEDISORB® Process

Microparticles comprising risperidone (of course, other drugs can be substituted for risperidone) were prepared at the 20-kilogram scale. The 20 Kg process (8 Kg of active agent and 12 Kg of polymer) provides a theoretical drug loading of the microparticles of 40% (8 Kg/20 Kg×100%).

The polymer solution was prepared by dissolving 12.0 Kg of Medisorb 7525 DL polymer (Alkermes, Inc., Blue Ash, Ohio) in 60 Kg of ethyl acetate (EMD Chemicals, Gibbstown, N.J.). The polymer was added to the solvent at 25° C. in a stainless steel reactor. The temperature of the tank was raised to 37° C. to facilitate dissolution. The vessel was agitated for at least 16 hours to dissolve the polymer. Once dissolved, the solution temperature was reduced to 25° C.

The drug solution was prepared by dissolving 8.0 Kg of risperidone base (Janssen Pharmaceutica, Beerse, Belgium) in 25.3 Kg of benzyl alcohol (Spectrum Chemicals, New Brunswick, N.J.) at 25° C. in a stainless steel reactor. The organic phase was prepared by adding the drug solution to the polymer solution at 25° C. in a stainless steel reactor and mixing for at least 15 minutes.

The continuous or aqueous phase was prepared by dissolving 6.0 Kg of polyvinyl alcohol (PVA) (DuPont, Wilmington, Del.) in 594.0 Kg hot (>60° C.) water for injection (WFI) in a stainless steel vessel to form a 1% solution. The vessel was agitated for at least 6 hours to dissolve the PVA. Once the PVA was dissolved, the temperature was reduced to 25° C. and 42.0 Kg of ethyl acetate (EMD Chemicals, Gibbstown, N.J.) was added, and mixed for at least 30 minutes to dissolve the ethyl acetate.

The two phases are combined using a static mixer, such as a ½″ Kenics static mixer available from Chemineer, Inc., North Andover, Mass. A total flow rate of 20 Kg/min generally provides microparticle size distributions with a mass median diameter (MMD) in the range of about 80-90 microns. The ratio of continuous phase to discontinuous phase is 4.5:1 (v/v).

The quench liquid is 2.5% solution of ethyl acetate and water-for-injection (WFI) at 5-10° C. The volume of the quench liquid is 0.25L per gram of batch size. The quench step is carried out for a time period greater than about 6 hours, with stirring of the microparticles in the quench tank.

After completion of the quench step, the microparticles are transferred to the collecting, de-watering, and drying device. The microparticles are rinsed using a chilled (approximately 5° C.) 300 Kg of a 25% ethanol solution.

The microparticles are then subjected to substantially complete intermediate drying. The microparticles are dried using vacuum and a dry nitrogen bleed. To avoid agglomeration, the temperature is maintained at less than 15° C. by chilling the dryer jacket and the feed nitrogen. Dryness is monitored by an absolute humidity probe, available from Vaisala, Inc., Woburn, Mass., in the vacuum line of the drying device. The substantially complete intermediate drying can be performed by drying under vacuum with a gas bleed or sweep (air, nitrogen or other dry gas) for a period in the range of approximately 16-48 hours.

The microparticles are then re-slurried in a re-slurry tank using a 25% ethanol solution (extraction medium) maintained at a temperature lower than the Tg of the microparticles. The temperature in the re-slurry tank is preferably in the range of 9° C. or less. The microparticles are then transferred back to the quench tank for washing for a time period of at least 6 hours with another extraction medium (25% ethanol solution) that is maintained at a temperature higher than the Tg of the microparticles. The Tg of the microparticles is about 18° C. (about room temperature), and the temperature of the extraction medium in the quench tank is greater than about 18° C., preferably 25° +/− 1° C.

The microparticles are transferred back to the collecting, de-watering, and drying device for de-watering and final drying. The final drying step is carried out in a manner similar to that described above for the intermediate drying step, but the temperature is warmed to greater than about 20° C. but below 40° C. Drying continues for a time period greater than about 16 hours.

Compaction Method for Manufacturing Extended Release Devices

Injectable microparticles comprising polymer and drug can be prepared using an efficient and facile single solvent process. PLG polymer and salt can be co-dissolved in a single solvent; (2) the solvent removed by vacuum drying or sublimation to form a polymer/drug matrix; (3) the matrix milled to produce a powder; (4) the resulting powder compacted to form a compressed matrix; and (5) the compressed matrix milled to form a dense, injectable microparticle formulation. Specifically, the drug loading can be about 30% or more (w/w) of the final weight of the microparticle composition. Solvents, for example, methylene chloride, acetone, dimethylsulfoxide (DMSO), acetonitrile, and ethyl acetate are suitable for use.

Suitable polymers include:

Lactide:Glycolide Ratio; Intrinsic Viscosity; End Group; Solvent

A 30% (w/w) 50:50; 0.75 dL/g; Acid end group; CH2CI2

B 30% (w/w) 75:25; 0.60 dL/g; Lauryl ester end group; CH2CI2

C 25 30% (w/w) 50:50; 0.61 dL/g; Lauryl ester end group; CH2CI2

The drug/polymer/solvent mixtures can be poured into either a polytetrafluoroethylene flat mold (approximately 1 inch×1 inch×½ inch deep) or a 3 inch diameter jar to form a film, for example. The films can be dried either in an FTS Dura-Dry Lyophilizer (Kinetic Systems, Inc., Santa Clara, Calif.) or in a vacuum oven. Films dried under various conditions including variation of maximum vacuum, ambient pressure, elevated temperature, ambient temperature, and drying time can be made.

The films can be milled using a 24-tooth Retsch Ultra Centrifugal Mill (Retsch, Inc., Newtown, Pa.) operating at 14,000 rpm. The collection pan is filled with liquid nitrogen prior to milling. The resulting powder, collected from the collection pan, is a flowable product that aids subsequent compaction steps. A portion of the powders produced by milling the films can be retained for analysis at this point. These powders can be retained for comparison with the powders made by the subsequent compacting and re-milling of the film powders described below.

A portion of the milled powders can be compacted using a Carver Model C Press (Carver, Inc., Wabash, Ind.) and either about ¼ inch or about ½ inch cylindrical dies. About 50 to about 300 milligrams of milled powder is filled into the dies and compacted at a machine setting of about 5000 pounds for about 30 seconds at room temperature to form pellets.

The compacted matrix is subsequently milled using a 24-tooth Retsch Ultra Centrifugal Mill (Retsch, Inc., Newtown, Pa.) operating at 14,000 rpm. The collection pan is filled with liquid nitrogen prior to milling. The final powder was collected from the collection pan and placed into vials for analysis.

The sustained release compositions described herein can also be prepared by any of emulsion, coacervation, and cryogenic microencapsulation techniques. The general process associated with each technique is described below.

Coacervation W/O/O Process

The coacervation process, also referred to herein as a water-oil-oil (W/O/O) process, requires formation of a water-in-oil emulsion with aqueous drug and organic polymer solutions. Oil, typically silicone oil, is then added to the water-in-oil emulsion to induce phase separation and to precipitate the polymer. The embryonic microparticles are then quenched in a solvent that removes the oil and polymer solvent. Drug is encapsulated in PLG polymer using a water-oil-oil (W/O/O) emulsion system. The initial embryonic microparticles were formed in a W/O/O inner emulsion step after which they are subjected to coacervation and hardening steps. The microparticles are collected, dried and filled into vials. Further details of each step in the complete process are set forth below.

A water-in-oil emulsion is created using sonication. The water phase of the emulsion contains dissolved drug and any optional excipients in water. The PLG phase contains polymer dissolved in methylene chloride.

Coacervation is induced by adding silicone oil at a controlled rate to the inner emulsion with agitation, forming embryonic microparticles. The embryonic microparticles formed are relatively soft and require hardening.

The embryonic microparticles are added to a heptane/ethanol solvent mixture with gentle agitation. The solvent mixture hardens the embryonic microparticles. After hardening for about one hour at about 3° C., the solvent mixture is decanted and pure heptane is added at 3° C. and mixed for about one hour.

After the hardening step, the microparticles are transferred and collected on a fine mesh pore-plate inside a drying chamber. A final heptane rinse of the hardening vessel is performed. The microparticles are dried with nitrogen gas over a four-day period with temperature ramping from about 3° C. to about 38° C.

In general, PLG is dissolved in methylene chloride. The inner water phase is prepared by dissolving the drug and excipients in water or an aqueous buffer. The aqueous solution is then injected into the polymer solution while probe sonicating. The resultant water/oil emulsion is then added to an emulsion reactor. Silicone oil (350 centiStokes) is slowly added to the reactor via peristaltic pump with stirring at about 1000 rpm. The mixture is then added to n-heptane. After stirring for about two hours, the microparticles are isolated by filtration and vacuum dried overnight.

Emulsion Process-W/O/W Process

The emulsion process is also referred to as a water-oil-water (W/O/W) process. Briefly, an aqueous solution of drug is dispersed in a polymer solution which is then emulsified in an outer aqueous phase (e.g., PVA). The microparticles are then hardened in an aqueous quench.

In a typical experiment, PLG (1.96 g) is dissolved in methylene chloride (22.5 g) and drug is dissolved in water (20 mg drug in 1.75 g water). The drug solution is then drawn up in a syringe and injected into the polymer solution while it is probe sonicated. The resultant W/O emulsion is then quickly added to an emulsion reactor containing 125 g aqueous 5% polyvinyl alcohol (PVA). The stir rate of the reactor is set to about 800 RPM. The mixture is stirred for about 1.5 minutes and then added to a water quench (2.8 L at 10° C.). After about two hours in the quench, the hardened microparticles are isolated by filtration and vacuum dried overnight.

Cryogenic Process

The cryogenic process used atomization to form droplets of polymer solution containing drug. Embryonic microparticles are then frozen in liquid nitrogen and the polymer solvent is removed through a subsequent ethanol extraction technique.

The cryogenic processing to produce microparticles includes two steps: (1) the production of a lyophilizate or dried drug substance; and (2) microencapsulation of the lyophilizate using a low-temperature, non-aqueous technique. Lyophilizates are formulated by atomizing a mixture of drug and excipient using a two-fluid nozzle, freezing the atomized droplets and drying the frozen droplets using lyophilization. It is understood that any suitable methods of drying known in the art can be employed. Specifically, frozen droplets are dried for about 7 days at a primary drying condition of −26° C. shelf and 96 mTorr chamber pressure followed by secondary drying for an additional 3 days at about 20° C. and 0 mtorr.

Drug containing microparticles can be produced with the cryogenic, non-aqueous process. Drug is suspended in an organic solution consisting of PLG dissolved in methylene chloride. This suspension is sonicated for about 4 minutes on ice, and then the suspension is atomized using a sonication nozzle and frozen by contacting with liquid nitrogen layered over a bed of frozen ethanol. The sample is warmed to −80° C. in order to allow microparticle hardening and extraction of solvent. The microparticles are then filtered and dried.

Solid/Oil/Water (S/O/W) and Solid/Oil/Oil (S/O/O) Processes

Solid drug can also be encapsulated using modified versions of the emulsion and coacervation processes described above. These modified processes are referred to solid/oil/water (S/O/W) and solid/oil/oil (S/O/O). For example, solid drug is suspended in methylene chloride containing PLG and sonicated for about four minutes on ice. Subsequent processing is conducted in a manner analogous to either the W/O/O or W/O/W methods.

Polymer:

Examples of specific PLG polymers suitable for use are listed below. All of the polymers employed in the following examples are set forth in the list and all listed polymers were purchased from Alkermes, Inc. and can be described as follows:

Polymer 2A: Poly(lactide-co-glycolide); 50:50 lactide:glycolide ratio; 12.3 kD Mol. Wt.; IV=0.15 (dL/g).

Polymer 2A-1: Poly(lactide-co-glycolide); 65:35 lactide:glycolide ratio; 16 kD Mol. Wt.; IV=0.19 (dL/g).

Polymer 2.5A: Poly(lactide-co-glycolide); 50:50 lactide:glycolide ratio; 25 kD Mol. Wt.; IV=0.24 (dL/g).

Polymer 3A: Poly(lactide-co-glycolide); 50:50 lactide:glycolide ratio; 47 kD Mol. Wt.; IV=0.38 (dL/g).

Polymer 3.5A: Poly(lactide-co-glycolide); 50:50 lactide:glycolide ratio; Mol. Wt., Not Determined; IV=0.42 (dL/g).

Polymer 4A: Poly(lactide-co-glycolide); 50:50 lactide:glycolide ratio; Mol. Wt. 45-64 kD; IV=0.45-0.47 (dL/g).

Polymer 4A-1: Poly(lactide-co-glycolide); 65:35 lactide:glycolide ratio; Mol. Wt. 53 kD; IV=0.43 (dL/g).

Modifications and variations of the invention will be obvious to those skilled in the art from the foregoing detailed description of the invention. Such modifications and variations are intended to come within the scope of the appended claims.

All patents, patent application publications and articles cited herein are incorporated by reference in their entirety.

1. A method for treating individuals at risk for CYP3A4 drug-drug interactions with an active agent metabolized by CYP3A4 comprising parenterally administering the active agent in a first extended release formulation in a first administration.

2. The method of claim 1 wherein the individual is being administered a CYP3A4 inhibitor.

3. The method of claim 2 wherein the CYP3A4 inhibitor is selected from the group consisting of fluconazole, omeprazole, saquinavir, quinine, ritonavir, nelfinavir, norfloxacine, sertraline, troleandomycin, diltiazem, delaviridine, nefazodone, zafirlukast, gestodene, amiodarone, cannabinoids, cimetidine, ciprofloxacin, clarithromycin, diethyldithiocarbamate, fluvoxamine, fluoxetine, mifepristone, grapefruit juice, indinavir, itraconazole, ketoconazole, metronidazole, mibefradil, miconazole, and erythromycin.

4. The method of claim 1 wherein the individual is need of treatment of a CNS disorder.

5. The method of claim 1 wherein the individual is under psychiatric treatment.

6. The method of claim 1 wherein the active agent is selected from the group consisting of antispychotics, antidepressants, serotonin reuptake inhibitors, neuroleptics, and opioids.

7. The method of claim 1 wherein the active agent is selected from the group consisting of aripiprazole, clozapine, rifampin, progesterone, propranolol, trazodone, prednisone, quinine, nitrendipine, ritonavir, R-warfarin, quinidine, taxol, ondansetron, nisoldipine, nimodipine, nifedipine, nicardipine, salmeterol, nelfinavir, triazolam, paclitaxel, verapamil, zolpidem, zaleplon, pravastatin, pimozide, haloperidol, caffeine, zileuton, terfenadine, vinblastine, saquinavir, methadone, testosterone, nefazodone, temazepam, tamoxifen, tacrolimus, simvastatin, sildenafil, sertraline, vincristine, chlorpheniramine, miconazole, dexamethasone, dapsone, cyclosporine, cyclophosphamide, cyclobenzaprine, codeine-N-demethylation, cocaine, clonazepam, clomipramine, clindamycin, diazepam, cisapride, diltiazem, cerivastatin, carbamazepine, cannabinoids, busulfan, buspirone, atorvastatin, astemizole, amlodipine, amitriptyline, alprazolalm, alfentanil, clarithromycin, frazodone, midazolam, mibefradil, lovastatin, losartan, lidocaine, lercadipine, lansoprazole, ketoconazole, isradipine, indinavir, imipramine, dextromethorphan, hydrocortisone, navelbine, finasteride, fexofenadine, fentanyl, felodipine, etoposide, ethosuximide, estrogens, estradiol, erythromycin, dronabinol, doxorubicin, donepezil, disopyramide, ifosfamide and analogs thereof.

8. The method of claim 1 wherein the active agent is aripiprazole.

9. The method of claim 1 wherein the active agent is administered by injection.

10. The method of claim 1 wherein the active agent is administered intramuscularly or subcutaneously.

11. The method of claim 1 wherein the first extended release formulation releases the active agent over a period of at least about 7 days.

12. The method of claim 1 wherein the first extended release formulation releases the active agent over a period of at least about 14 days.

13. The method of claim 1 further comprising a second administration of an active agent in a second extended release formulation at least about 7 days after the first administration.

14. The method of claim 1 further comprising a second administration of an active agent in a second extended release formulation at least about 14 days after the first administration.

15. The method of claim 1 further comprising a second administration of an active agent in a second extended release formulation at least about 17 days after the first administration.

16. The method of claim 15 wherein the second extended release formulation is substantially similar to the first extended release formulation.

17. The method of claim 1 wherein the first extended release formulation comprises a biodegradable polymer and the active agent.

18. The method of claim 1 wherein the first extended release formulation comprises a polylactide and the active agent.

19. The method of claim 1 wherein the first extended release formulation comprises a polylactide-co-glycolide and the active agent.

20. The method of claim 19 wherein the active agent is aripiprazole.

21. A method for preventing adverse drug reactions in individuals with an active agent metabolized by CYP3A4 comprising parenterally administering the active agent in a first extended release formulation in a first administration.
